(19) United States
(12) Patent Application Publication
(10) Pub. No.: US 2002/0087116 A1
(43) Pub. Date: Jul. 4, 2002

(54) Patient scheduling techniques for an implantable medical device

(75) Inventor: Jerome T. Hartlaub, St. Paul, MN (US)

Correspondence Address: BANNER & WITCOFF, LTD., TEN SOUTH WACKER DRIVE, SUITE 3000, CHICAGO, IL, 60606, US

(73) Assignee: Medtronic, Inc., Minneapolis, MN

(21) Appl. No.: 10/002,669

(22) Filed: Oct. 31, 2001

Related U.S. Application Data
(60) Provisional application No. 60/259,022, filed on Dec. 29, 2000.
(60) Provisional application No. 60/259,008, filed on Dec. 29, 2000.
(60) Provisional application No. 60/259,115, filed on Dec. 29, 2000.
(60) Provisional application No. 60/259,116, filed on Dec. 29, 2000.

(51) Int. Cl. A61M 31/00; A61M 37/00

(52) U.S. Cl. 604/65; 604/288.01

(57) ABSTRACT

Disclosed is a method and apparatus for scheduling a patient for an appointment with a caregiver, for example, when an implantable device needs to be refilled for drug. The date scheduling of a patient visit to a clinic is automatically arranged and communicated to caregivers and the patient based on a scheduling algorithm. The system includes an implantable drug delivery device having stored therein at least one drug, a drug monitor module monitoring drug usage and drug levels, an external device in telemetric communication with the implantable drug delivery device and having a drug scheduling module for determining whether patient should visit with his/her caregiver. If it is determined that the patient should visit his/her caregiver, the external device will contact various entities involved in the healthcare management of the patient to schedule an appointment. The various entities include, for example, an insurance provider, a pharmacy, a hospital, a caregiver, a physician, and a device manufacturer.

This application claims priority to provisional U.S. Provisional Application Ser. No. 60/259,022, filed Dec. 29, 2000, which is incorporated herein by reference in its entirety.

This patent application is related to the following co-pending patent applications, each of which having the same named inventor and filing date as the present application:

a. U.S. Patent Application Ser. No.______, entitled “Non-Conformance Monitoring And Control Techniques For An Implantable Medical Device,” having attorney reference no. 011738.00045 (based on U.S. Provisional Application Ser. No. 60/259,008, filed Dec. 29, 2000);

b. U.S. Patent Application Ser. No.______, entitled “Drug Management Techniques For An Implantable Medical Device,” having attorney reference no. 011738.00044 (based on U.S. Provisional Application Ser. No. 60/259,115, filed Dec. 29, 2000); and

c. U.S. Patent Application Ser. No.______, entitled “Therapy Management Techniques For An Implantable Medical Device,” having attorney reference no. 011738.00043 (based on U.S. Provisional Application Ser. No. 60/259,116, filed Dec. 29, 2000).

Each of these related co-pending patent applications are incorporated herein by reference in their entireties.

### FIELD OF THE INVENTION

This invention relates to implantable drug delivery devices such as implantable drug delivery devices, and more particularly relates to automated patient scheduling systems and methods for implantable drug delivery devices.

### BACKGROUND OF THE INVENTION

The medical device industry produces a wide variety of electronic and mechanical devices suitable for use outside and inside the body for treating patient disease conditions. Devices used outside the body are termed external while devices used inside the body are termed implantable and include therapeutic substance infusion devices such as implantable drug pumps. Clinicians use medical devices alone or in combination with therapeutic substance therapies and surgery to treat patient medical conditions. For some medical conditions, medical devices provide the best, and sometimes the only, therapy to restore an individual to a more healthful condition and a fuller life. Implantable therapeutic substance infusion devices can be used to treat conditions such as pain, spasticity, cancer, and a wide variety of other medical conditions.

Implantable medical devices have important advantages over other forms of therapeutic substance administration. For example, oral administration is often not workable because the systemic dose ofthe substance needed to achieve the therapeutic dose at the target sight may be too large for the patient to tolerate without very adverse side effects. Also, some substances simply will not be absorbed in the gut adequately for a therapeutic dose to reach the target sight. Moreover, substances that are not lipid soluble may not cross the blood-brain barrier adequately if needed in the brain. In addition, infusion of substances from outside the body requires a transcutaneous catheter, which results in other risks such as infection or catheter dislodgement. Further, implantable medical devices avoid the problem of patient noncompliance, namely the patient failing to take the prescribed drug or therapy as instructed.

Implantable medical devices are often used in conjunction with various computer and telecommunication systems and components. Information obtained by the implantable medical device may be stored and subsequently transmitted to a physician or patient caregiver or a database on demand or automatically. Many ways ofusing the information are known including decision making to provide optimum medical care to the person with the medical condition.

An implantable therapeutic substance infusion device such as an implantable drug delivery device is implanted by a clinician into a patient at a location appropriate for the therapy that interferes as little as practicable with normal patient activity. This location is typically a subcutaneous region in the lower abdomen. The proximal or near end of the infusion catheter is connected to the drug pump infusion outlet. The catheter is simply a flexible tube with a lumen typically running the length of the catheter. The distal or far end of the catheter is positioned to infuse a drug or drug combination to a target site in the patient. Target sights in the body included but are not limited to an internal cavity, any blood vessel, any organ, or other tissue in the body. The drug or other therapeutic substance flows from the pump via the lumen in the catheter at a programmed infusion rate to treat the disease condition. The pump typically includes an expansible reservoir for containing a refillable supply of drug. For example, U.S. Pat. Nos. 4,692,147 (Duggan) and 5,445,616 (Kratoska et al) disclose types of implantable pumps that can be used.

Examples of diseases that are treatable include spasticity and chronic intractable pain. To treat spasticity, the distal tip of the catheter is typically surgically positioned in the intrathecal space of the patient's spinal column. Drug flows out of the distal tip into the cerebral spinal fluid where it baths the spinal cord. By virtue of molecular action on nervous tissue in the spinal cord, the patient's spasticity symptoms are dramatically reduced and the patient becomes much more comfortable and competent. Pain patients are treated in much the same way.

The infusion rate of the drug pump is typically programmed to be variable over time. The rate is usually controlled by certain components in the pump. The controlled infusion rate is often further set by using an external device or programmer to transmit into the pump, instructions for the controlled infusion. The controlled infusion may be variable as time passes according to the needs of the patient. The instructions provided to the pump to control the infusion rate of the drug pump are typically determined by a medical person. In some cases the patient is able to provide the instructions to the pump via an external patient-programming device. In contrast, fixed rate pumps usually cannot be programmed and are only capable of constant infusion rate.

Eventually, the drug delivery device will deplete its drug reserve and will require refill with more drug. To avoid cessation of drug infusion, many implantable drug pumps are configured so the pump can be replenished with drug through a refill port or septum while the pump is implanted. In some pumps, various techniques are used to warn the patient or caregiver that the drug pump reservoir is nearly empty. One technique is the pump will provide a modest audio warning sound when the pump drug reservoir is nearly empty and the pump is about to cease normal infusion.

Typically, when the drug pump requires drug refill, a trained medical practitioner, typically a nurse or a doctor, must refill the device. Before refilling the device, several procedures are required. First, the patient must schedule an appointment with the trained medical practitioner to refill the implanted device. Then the trained medical practitioner must coordinate with the pharmacy to ensure that the drug is available. The trained medical practitioner also may need to coordinate with the patient's managed care company to ensure payment for the drug refill. Only after all of these processes are accomplished, the patient may then visit the trained medical practitioner to have the drug delivery device refilled. All of these procedures typically are handled manually and are fraught with inefficiencies and sometimes inaccuracies.

One such inefficiency is that the patient is sometimes not aware of when the implanted device needs to be refilled. Occasionally, the patient will learn that the device needs refilling when the pump is entirely depleted of drug. Until the patient meets with the physician, the patient must endure a time period where the patient cannot receive any drug treatment therapy from the device. Of course, if the drug delivery device delivered a predetermined and steady dosage of drug to the patient, the device would be depleted at known periods. This is not always the case, however, since many devices are capable of delivering drug at varying levels depending upon the patient's needs or are capable of allowing the patient to control the infusion rate.

In addition to requiring pump refills, the patient may also need to make an appointment for some other purpose such as pump diagnostic or pump maintenance.

It is therefore desirable to provide an improved implantable drug delivery system that allows patients to obtain drug refills of their implanted pump or obtain pump servicing on a timely basis, avoiding the risk of stoppage of drug delivery due to unpredictable events.

### BRIEF SUMMARY OF THE INVENTION

The present invention is an automated scheduling system for implantable drug delivery devices. The overall system generally includes an implantable drug delivery device, an external device having a drug scheduling module in bidirectional communication with the implantable device, a computing network coupled to the external programmer and various entities involved in the healthcare management of the patient. The drug scheduling module receives various information to determine whether and when the implanted device should be refilled or serviced. The drug scheduling module receives as inputs drug usage information from the implanted device, drug management instructions, drug management data, and pump manufacturer requirements information. Based on these inputs, if is determined that the implanted device needs to be refilled or serviced, the drug scheduling module will communicate with the various healthcare entities to schedule an appointment for the patient to have his/her device refilled or serviced. Such entities may include, for example, an insurance provider, a pharmacy, a hospital, a caregiver, a physician, and/or a device manufacture and may have a corresponding scheduling module.

The date scheduling of patient visits to a clinic or visits by a nurse for pump refill are automatically arranged and communicated to caregivers and the patient by an appointment scheduling module and based on a scheduling algorithm. The scheduling algorithm considers various variables in scheduling an appointment including, drug volume remaining, predicted/calculated drug usage rate, drug life, etc. For example, the date, time, and place for refill and follow-up could be determined and communicated to the various entities. These entities may also initiate a scheduling routine that the system automatically implements and verifies.

In alternative embodiments, the drug scheduling module maybe implemented in other parts of the overall system for drug scheduling including, for example, in the implantable drug delivery device or on a server accessible over the computing network.

The objects, advantages novel features, and the further scope of applicability of the present invention will be set forth in the detailed description to follow, taken in conjunction with the accompanying drawings, and in part will become apparent to those skilled in the art upon examination of the following, or may be learned by practice of the invention. The objects and advantages of the invention may be realized and attained by means of the instrumentalities and combinations particularly pointed out in the appended claims.

### BRIEF DESCRIPTION OF THE DRAWINGS

These and other advantages and features of the invention will become apparent upon reading the following detailed description and referring to the accompanying drawings in which like numbers refer to like parts throughout and in which:

FIG. 1 is a schematic block diagram of an overall system for scheduling management of an implantable drug delivery device in accordance with a preferred embodiment of the present invention.

FIG. 2 is a diagrammatic view of a drug delivery device for use with the present invention as implanted within a patient.

FIG. 3 illustrates a typical position in a patient of an implantable drug delivery device with a catheter implanted at or near a spinal cord.

FIG. 4 illustrates another typical position in a patient of an implantable drug delivery device with a catheter implanted at or near a brain.

FIG. 5 depicts the implantable drug delivery device.

FIG. 6 shows an implantable pump communicating via telemetry with an external handheld programming device.

FIG. 7 is a diagrammatic view of an exemplary implantable drug delivery device for use with the present invention depicting the various layered components of the device.

FIG. 8 shows a block diagram of an implantable drug delivery device embodiment for use with the present invention.

FIG. 9 is a schematic block diagram of the electronic modules of the implantable drug pump in accordance with a preferred embodiment of the present invention.

FIG. 10 is a schematic block diagram of the drug scheduling module of the implantable drug pump in accordance with a preferred embodiment of the present invention.

FIG. 11 is a flow chart depicting the process for determining whether drug in the implantable pump needs to be refilled in accordance with a preferred embodiment of the present invention.

### DETAILED DESCRIPTION OF THE INVENTION

In accordance with the present invention, an implantable drug delivery device is equipped with automated management control capabilities. Although not required, part of the invention will be described in part in the general context of computer-executable instructions, such as program modules. Generally, program modules include routines, programs, objects, scripts, components, data structures, etc. that perform particular tasks or implement particular abstract data types. Program modules may be part of a single software program, may be implemented as separate software programs, or may be part of hardwired devices having various electronic components for carrying out the desired functionality.

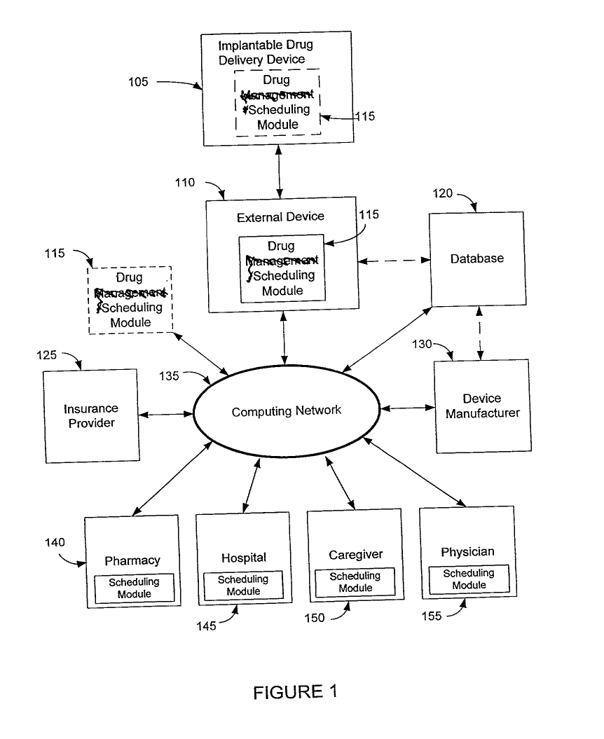

FIG. 1 is a schematic block diagram of an overall system for drug scheduling management of an implantable drug delivery device in accordance with a preferred embodiment of the present invention. The overall system generally includes an implantable drug delivery device 105 implantable within a patient, an external device or programmer 110 having a drug scheduling module 115 in accordance with a preferred embodiment of the present invention, a database 120, and a computing network 135 such as the Internet coupled to various entities involved in the healthcare management of the patient. Such entities may include, for example, an insurance provider 125, a pharmacy 140, a hospital 145, a caregiver 150, a physician 155, and/or a device manufacture 130. In alternative embodiments, the drug scheduling module 115 may be implemented in other parts of the overall system for drug scheduling management including, for example, in the implantable drug delivery device 105, or on a server accessible over the computing network 135. Further details of the drug scheduling module are discussed in further detail herein. The implantable drug delivery device 105 is coupled to be in bidirectional communication with the external device 110 via telemetry. The external device 110 may be any computing device capable of communicating with the implantable drug delivery device 105, including for example, a physician programmer, a patient programmer, a screening device, a data acquisition device, and the like. The bidirectional communications may be of any type of telemetry including RF.

The external device 110 is preferably coupled to the computing network 135 for communicating with various healthcare entities essential to the management ofthe treatment therapy of the patient. Also coupled to the network 135 and in communication with the external device 110 is the database 120 storing drug management information relating to the patient. The computing network 135 may be, for example, a public network such as the Internet, an intranet, an extranet, or a private network. The computing network 135 enables the external device 110 to communicate with the various healthcare entities and the database 120.

The external device 110 may be coupled to the computing network 135 either directly through a modem or may be networked to a personal computer that is coupled to the computing network 135 through known techniques. The various other entities 125, 130,140-155 are preferably coupled to the computing network 135 via a general-purpose computing device. The computing devices used by these entities preferably have installed therein a software application that communicates with the drug scheduling module to perform the various scheduling functions to be performed.

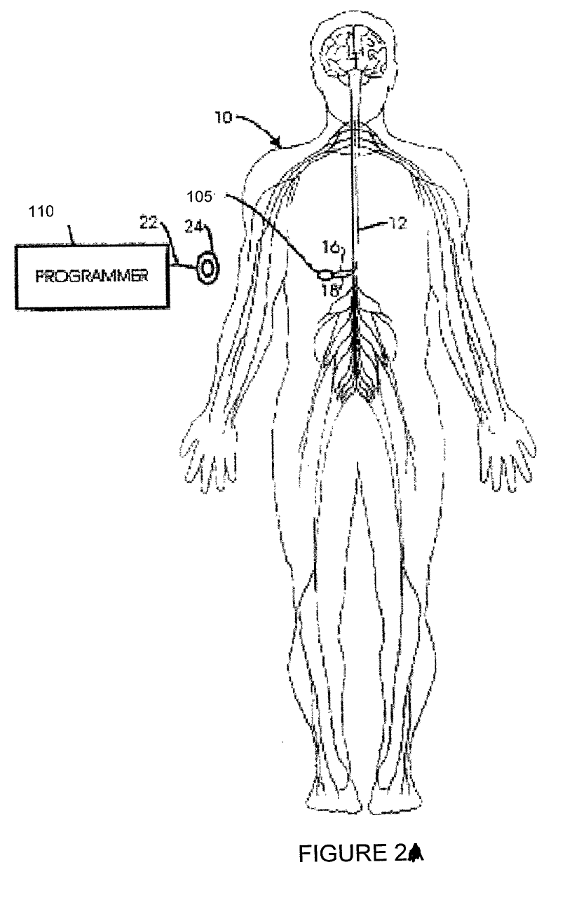

As discussed, implantable drug delivery devices are generally known in the art. U.S. Pat. Nos. 4,692,147 (Duggan) and 5,445,616 (Kratoska et al), for example, illustrate the general features of these devices. FIG. 2 is a diagrammatic illustration of an exemplary implantable drug delivery device 105 for use with the present invention. The system includes the device 105 that may be implanted below the skin of a patient 10 in the abdomen or any other location of the body. The device 105 is typically a pump that delivers drug to a catheter 16/18that is positioned to deliver the drug to specific infusion sites within the patient's body (in this case, the spinal cord 12). The distal end of the catheter 16/18terminates in a cylindrical hollow tube having a distal end implanted into a portion of the body by conventional surgical techniques. The catheter 16/18is joined to the implanted device 105 in the manner shown, and may be secured to the device 105 by, for example, screwing the catheter 16/18onto a catheter port of the device 105.

The implantable system 105 may include one or more sensors to provide closed-loop feedback control of the drug delivery system to provide enhanced results. Sensors can be used with a closed loop feedback system to automatically determine the level of treatment therapy necessary to alleviate the symptoms of the disorder being treated. The sensor is attached to or implanted into a portion of a patient's body suitable for detecting symptoms of the disorder being treated, such as a motor response or motor behavior. The sensor is adapted to sense an attribute of the symptom to be controlled or an important related symptom. For movement disorders that result in abnormal movement of an arm of the patient, such as an arm, the sensor may be a motion detector implanted in the arm. For example, the sensor may sense three-dimensional or two-dimensional motion (linear rotational or joint motion), such as by an accelerometer. One such sensor suitable for use with the present invention is described in U.S. Pat. No. 5,293,879 (Vonk). The sensor also may be placed in the implantable drug delivery device, for example, to sense drug levels. Those skilled in the art will appreciate that any type of sensor may be utilized with the present invention. The output ofthe sensor may be coupled by a cable or via telemetry to the input of an analog to digital converter within the implantable drug delivery device. Alternatively, the output of an external sensor would communicate with the implantable drug delivery device through a telemetry downlink.

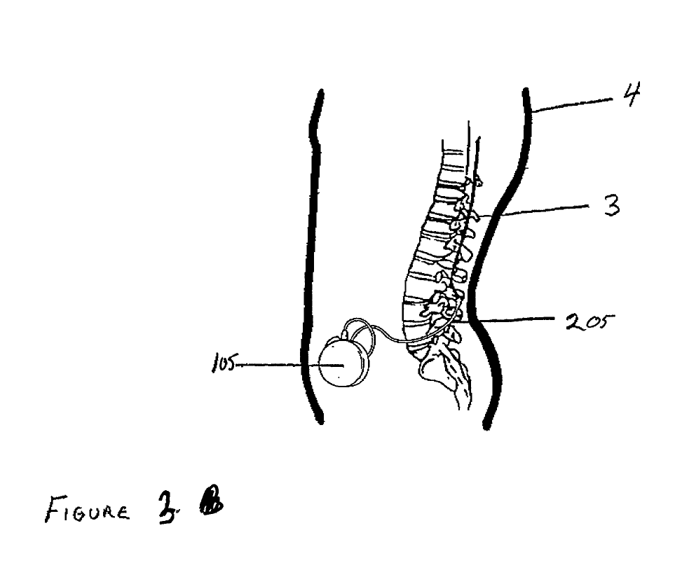

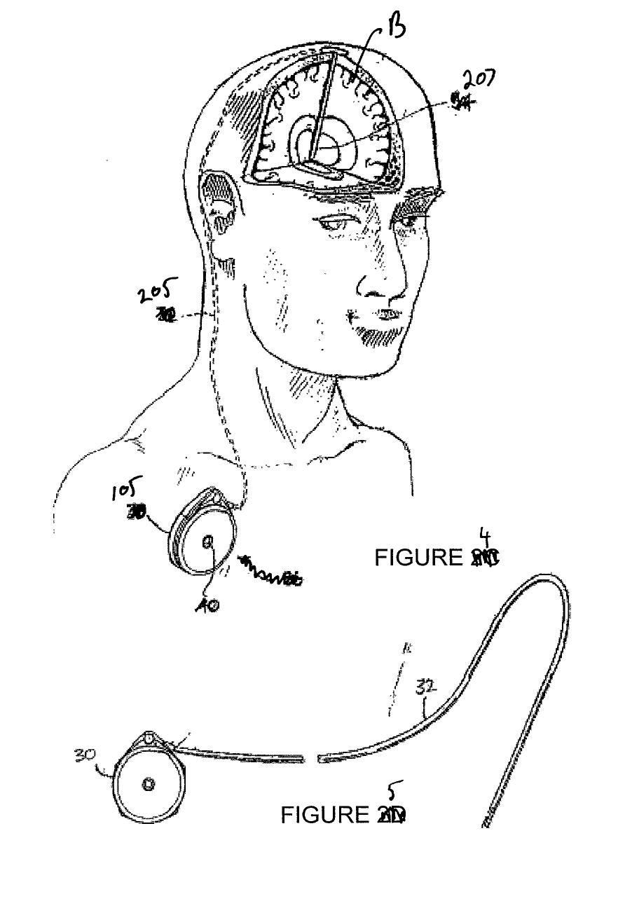

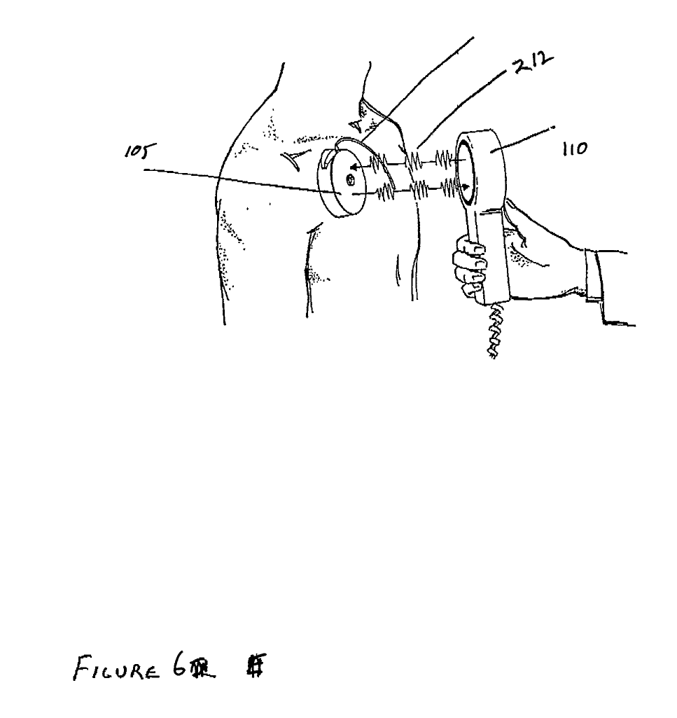

The implantable drug delivery device 105 can be used for a wide variety of therapies to treat medical conditions (also known as medical indications) such as pain, spasticity, cancer, and many other medical conditions. The implantable drug delivery device 105 is typically implanted by a clinician, such as a surgeon, using a sterile surgical procedure performed under local, regional, or general anesthesia. Before implanting the therapeutic substance infusion device, a catheter is typically implanted with the distal end position at the desired therapeutic substance infusion site and the proximal end tunneled to the location where the therapeutic substance infusion device is to be implanted. The implantable therapeutic substance infusion device is generally implanted subcutaneously about 2.5 cm (1.0 inch) beneath the skin where there is sufficient subcutaneous tissue to support the implanted system. As one example, FIG. 3 illustrates the implantable drug delivery device 105 coupled to catheter 205, both of which are under the surface of the skin 4. The catheter 205 is positioned with its distal tip in the intrathecal space of the spinal column 3. As another example, FIG. 4 shows the implantable drug delivery device 105 for infusion of drug into to brain B. The device 105 is coupled to catheter 205 with a distal end terminating within the brain B. FIG. 5 illustrates the various components of the implantable drug delivery device 105 that are implanted within the patient 10.

Once the therapeutic substance infusion device is subcutaneously implanted into the patient, the incision can be sutured closed and the therapeutic substance infusion device can begin operation. The implantable drug delivery device 105 operates to infuse a therapeutic substance at a programmed rate into a patient. The therapeutic substance is a product or substance intended to have a therapeutic effect such as pharmaceutical compositions, genetic materials, biologics, and other substances. Pharmaceutical compositions are chemical formulations intended to have a therapeutic effect such as intrathecal antispasmodics (e.g., balcofen), pain medications, chemotherapeutic agents, and the like. Pharmaceutical compositions are often configured to function in an implanted environment with characteristics such as stability at body temperature to retain therapeutic qualities, concentration to reduce the frequency of replenishment, and the like. Genetic materials are substances intended to have a direct or indirect genetic therapeutic effect such as genetic vectors, genetic regulator elements, genetic structural elements, DNA, and the like. Biologics are substances that are living matter or derived from living matter intended to have a therapeutic effect such as stem cells, platelets, hormones, biologically produced chemicals, and the like. Other substances are substances intended to have a therapeutic effect yet are not easily classified such as saline solution, fluoroscopy agents, and the like. As used herein, the term drug shall refer generally to any therapeutic substance.

The therapeutic substance can be replenished in some embodiments of the implanted therapeutic substance infusion device by inserting a non-coring needle connected to a syringe filled with therapeutic substance through the patient's skin into a septum and into a reservoir in the therapeutic substance infusion device to fill the implanted device reservoir. Refill kits are available which include the drug and all other necessary equipment needed for the medical attendant to refill the pump.

A therapeutic substance bolus can be administered by a clinician, in some embodiments, by inserting a non-coring needle connected to a syringe into a catheter access port. This procedure can be used for several other reasons including reopening the catheter if it becomes occluded or to withdraw a sample of cerebral spinal fluid for investigative purposes.

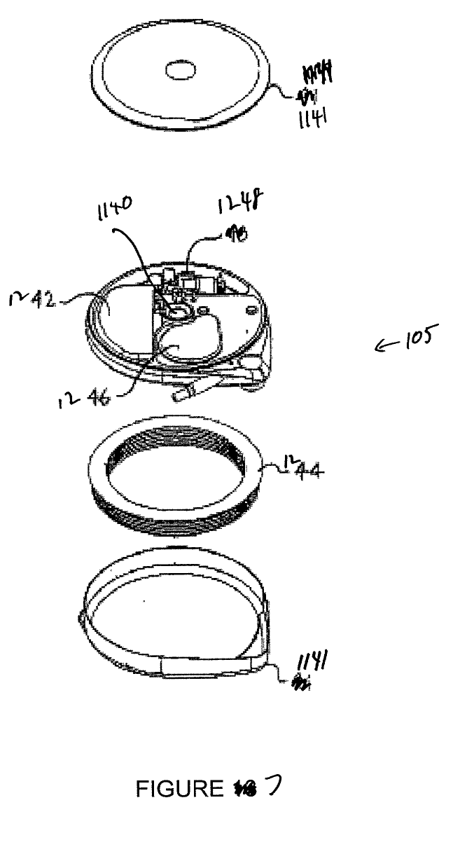

FIG. 6 illustrates a typical pump programming technique. An external device, a handheld programming device 110 in this embodiment, transmits and receives radio frequency signals 212 to and from the implantable drug delivery device 105. The radio frequency signals 212 sent to the pump, often called the downlink signal, contain the programming instructions needed by the implantable drug delivery device 105 for it to correctly infuse a drug into the patient from its drug reservoir. Many other types of information may be sent to the pump including requests for information residing in the pump in accordance with the present invention (discussed herein).

The implantable drug delivery device 105 may continuously or periodically store various types of information including, for example without limitation, pump diagnostics, drug delivery information, batter life, etc. Further, the implantable drug delivery device 105 may receive information from various sensors inside the pump or information from sensors integral with the catheter, thereby obtaining physiological information about the patient. Even further, the implantable drug delivery device 105 may store historical data about the drug infusing profile, patient requests for more drug or other such information.

Such information stored in the pump may be valuable to the treating physician and/or the medical device supplier and can be retrieved from the pump. In particular, the information stored in the implantable drug delivery device 105 may be retrieved in response to a request by the pump from the programming device 110. After the request is received and processed in the implantable drug delivery device 105, the implantable drug delivery device 105 prepares the requested information and sends it to the programming device 110, sometimes called uplink data. The pump information received by the programming device 110 is processed and converted to intelligible data for clinical or technical use. This intelligible data can be used for many purposes including management of the pump performance, management of the patient therapy, and/or other medical or record-keeping purposes.

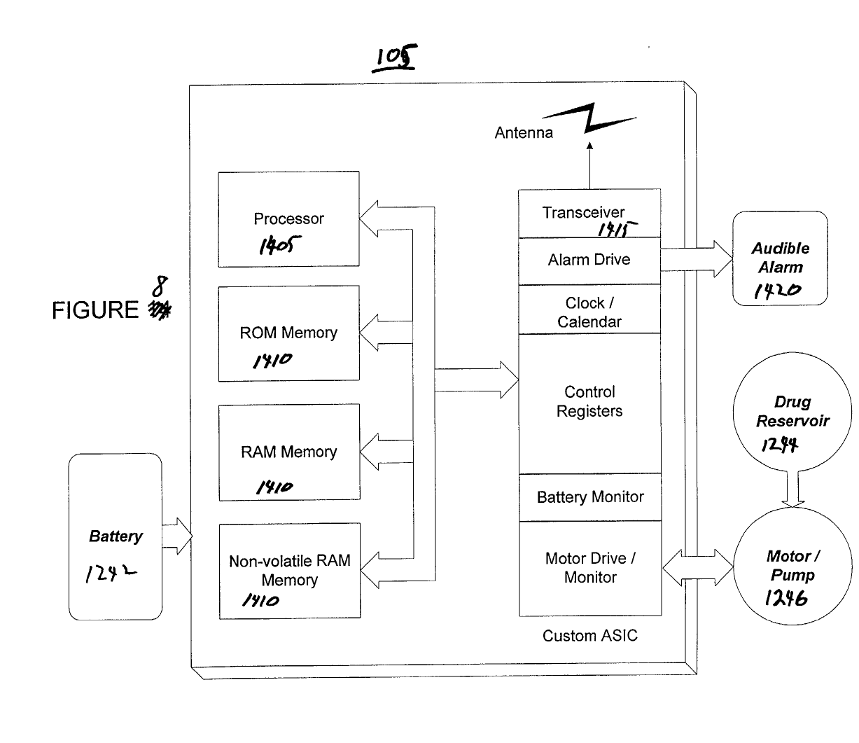

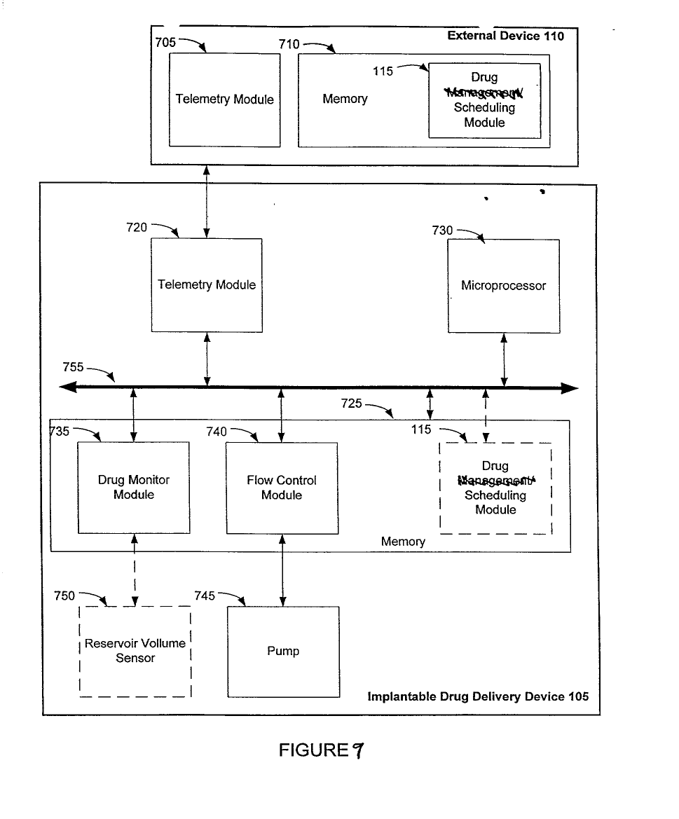

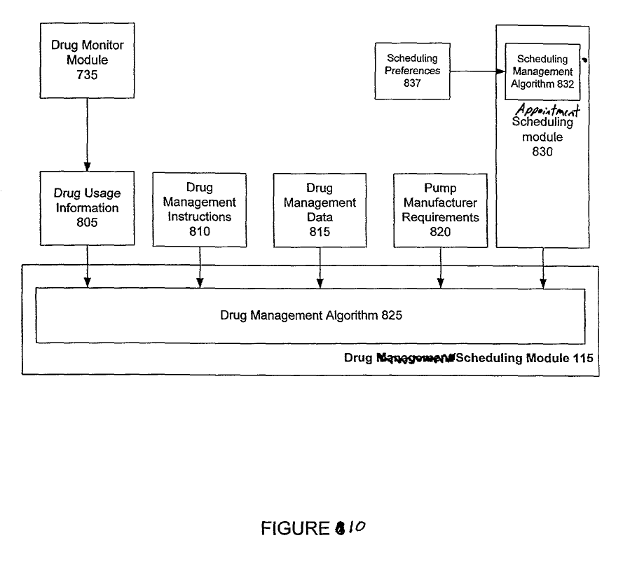

Referring back to the embodiment of the implantable drug delivery device, the present invention may be implemented for use any number of such devices. FIG. 7 show one such example of the implantable drug delivery device 105 and FIG. 8 shows a block diagram of the implantable drug delivery device 105. The implantable drug delivery device 105 generally comprises a housing 1141, a power source 1242, a therapeutic substance reservoir 1244, a therapeutic substance pump 1246, and electronics 1248. The housing 1141 is manufactured from a material that is biocompatible and hermetically sealed such as titanium, tantalum, stainless steel, plastic, ceramic, and the like. The power source 1242 is carried in the housing 1141. The power source 1242 is selected to operate the therapeutic substance pump 1246 and electronics 1248 such as a lithium ion (Li+) battery, capacitor, and the like.

The therapeutic substance reservoir 1244 is carried in the housing 1141. The therapeutic substance reservoir 1244 is configured for containing a therapeutic substance. The therapeutic substance reservoir 1244 may be refilled with therapeutic substance while implanted via port 1140. The therapeutic substance pump 1246 is carried in the housing 1141. The therapeutic substance pump 1246 is fluidly coupled to the therapeutic substance reservoir 1244 and electrically coupled to the power source 1242. The therapeutic substance pump 1246 is a pump that is sufficient for infusing therapeutic substance such as a piston pump, a peristaltic pump that can be found in the SynchroMed® Infusion System available from Medtronic, Inc., or a pump powered by a stepper motor, an AC motor, a DC motor, an electrostatic diaphragm, a piezoelectric diaphragm, a piezoelectric motor, a solenoid, a shape memory alloy, and the like.

The electronics 1248 are carried in the housing 1141 and coupled to the therapeutic substance pump 1246 and the power source 1242. The electronics 1248 include a processor 1405, memory 1410, an infusion program in memory, and transceiver circuitry 1415. The processor 1405 can be an Application Specific Integrated Circuit (ASIC) state machine, a gate array, controller, and the like. The electronics 1248 are configured to control the infusion rate of the therapeutic substance pump 1246 and can be configured to operate many other features such as patient alarms 1420 and the like. The infusion program resides in memory and is capable of being modified once the implantable drug deliver device is implanted. The transceiver circuitry 1415 is coupled to the processor 1405 for externally receiving and transmitting therapeutic substance infusion device information.

As discussed, the present invention is implemented in part as computer-executable instructions, such as program modules. In a preferred embodiment as discussed herein, some of the features of the present invention are implemented within a drug scheduling module 115. The implantable device 105 would provide via telemetry the necessary information for the external device 110 to provide the drug scheduling management functionality of the present invention. In the embodiment where the drug scheduling module 115 is within the implantable device 105, it may be found in the electronic module 1242 or 32.

Referring to the schematic block diagram of FIG. 9, the implantable device 105 includes various electrical and software components including a microprocessor 730, a flow control module 740 for controlling the flow of drug from the reservoir to the infusion port, a telemetry module 720 for providing bidirectional communication between the implantable device 105 and the external device 110, a memory 725 for storing the various software modules for use with the present invention, a drug monitor module 735, and (optionally) a drug scheduling module 115. The drug monitor module 735 provides one or more drug usage parameters that determine the amount of drug remaining in the implantable device 105. Drug usage parameters monitored by the drug monitor module 735 may include, for example and without limitation, the quantity drug consumed by the patient, the rate in which the drug is being consumed by the patient, and the estimated date that the drug in the pump will be exhausted based on the previous two parameters. Drug usage parameters maybe determined, for example, by way of a pump reservoir sensor 750 that senses the amount of drug remaining in the pump reservoir. For example, the pump reservoir sensor 750 disclosed in U.S. Pat. No.____________. having Application Ser. No. 09/070,255, filed Apr. 30, 1998, and entitled “Reservoir Volume Sensor”, may be used.

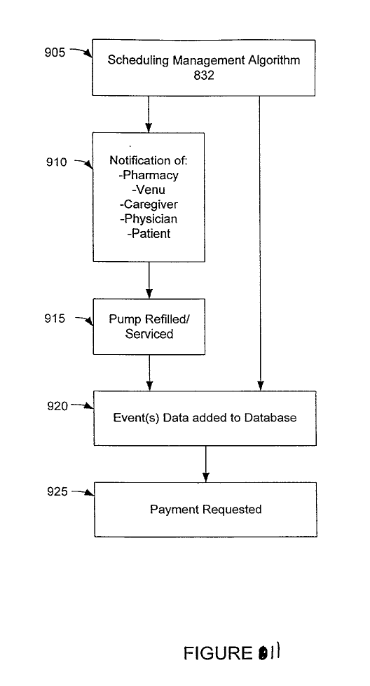

The external device 110 generally includes a telemetry module 705 and a memory 710 for storing various software applications and modules for use with the present invention. Stored within the external device 110 is the drug scheduling module 115. The drug scheduling module 115 gathers data regarding the implantable device 105 to determine whether the drug level in the implantable device 105 is low and thereby needs to be replenished. The drug scheduling module 115 may also gather diagnostic data regarding the implantable device 105 to determine whether the device requires servicing. As shown in the block diagram of FIG. 10, the data regarding the implantable device 105 that the drug scheduling module 115 uses to make its determination include, for example, drug usage information 805 from the drug monitor module 735, drug management instructions 810, and pump manufacturer requirements 820. Drug usage information 805 provides information regarding the amount of drug remaining in the implantable device 105 and the rate at which the drug is being depleted. Drug management instructions 810 provide information about the particular requirements for refilling the drug and the particular requirements of the patient. For example and without limitation, the drug management instructions 810 may include: the number of days that the replacement drug must be ordered before an estimated drug exhaustion date, to order a drug delivery device refill kit, to notify primary care physician of the drug order, to notify the specialty care physician of the drug order, to notify the drug pharmacy to order the drug from the drug manufacturer, to notify the patient's employer of drug order, to deliver the drug to a specified location, and to bill the drug to a specified payer. The pump manufacturer requirements 820 provides a continuous real time input to the drug scheduling module 115 to allow the pump manufacturer to specify different reservoir levels for filling based on, for example, more knowledge about the pump performance. For example, the pump manufacturer requirements 820 may specify the drug level that the drug scheduling module 115 should decide that a pump refill is needed. A specified level for initiating a refill could be different depending on the type of drug as well as changes to the reservoir volume depending upon the type of pump used.

The drug scheduling module 115 also receives drug management data 815 to determine drug order information. The drug management data 815 may include, for example and without limitation, the name of the drug manufacturer, the date the drug was manufactured, the name of the pharmacy carrying the drug.

Still referring to FIG. 10, the drug scheduling module 115 includes a drug management algorithm 825 that serves to forecast when the next refill of the pump reservoir is required. The drug management algorithm 825 schedules a refill by virtue of comparing the drug usage information 805 with the drug management instructions 810, the pump manufacturer requirements 820, and the drug management data 815 to determine whether and when refill should be ordered. The drug management algorithm 825 considers these various variables that would be a part of this forecast including particularly, but not limited to, the total amount of drug used to date by the patient, the drug infusion profile of the patient, the average infusion rate programmed by the physician, and a profile of recent drug usage by the patient. The profile of recent usage (e.g., over the past several days) may be used as an indicator of the usage rate until the pump reservoir contents are totally infused and the reservoir is empty of drug. Thus, various preferences may be pre-set with the drug management algorithm 825 including, for example, the average drug usage rate as well as the number of days prior to the reservoir empty condition before which the patient should go to a clinic for pump refill.

Appointment scheduling module 830 has a scheduling management algorithm 832, which performs the function of arranging an appointment for the patient to refill the pump. There are many scheduling preferences 837 or factors that contribute to optimum automated scheduling capability. Such preferences 837 include but are not limited to a number of days prior to pump reservoir drug depletion before the pump is refilled, the date and time preferences of the pump refill technician or physician, the date and time and place preferences ofthe patient and the caregiver(s), the date and time and availability ofthe clinic rooms, the proximity of the clinic to the patient and the pump refill technicians, holiday and work schedules, the pharmacists delivery timeline, and the back-up hospital staff availability. These and other preferences 837 could be manually provided to the appointment scheduling module 830 or could be provided as needed via the computing network 135. The scheduling preferences 837 would be accessed each time the scheduling algorithm 832 was enabled in order to initiate the automated scheduling task.

In the scheduling system, any ofthe preferences 837 could be reset by any ofthe contributors to the preferences in such a way as to keep the database current with all factors associated with the scheduling management algorithm 832. These preferences 837 could be adjusted remotely from either a telephone or a web-connected personal computer. The scheduling management algorithm 832 would also record the acceptance of the resulting schedule in the delivery of the pharmaceutical agent as well as all other parameters (personnel availability, room availability) so that the scheduling task would not be complete until all the entities being scheduled have acknowledged acceptance. If acceptance would not be acknowledged, the scheduling algorithm would continue to search for an optimum schedule based on the preferences established.

Scheduling management algorithm may be called for more than just for refilling the pump. For example, if it is determined that the therapy is not as effective as desired or expected, the scheduling management algorithm 832 may be called upon to arrange for the patient to be seen by a physician. See U.S. Patent Application Ser. No.______, entitled “Drug Management Techniques for an Implantable Device,” filed on the same date as the present application, having the same named inventor and having attorney reference no. 11738.00044. As used herein pump refill or servicing shall include any number of pump maintenance matters, including but not limited to, software updates, software modifications, pump refills, and power supply recharge.

The scheduling management algorithm 832 notifies the various entities that need to know about the upcoming appointment including the patient, the treating physician, the caregiver, a meeting place such as a clinic or hospital, as well as the pharmacy if drug needs to be ordered. These entities may be contacted, for example, via the computing network 135 (as shown in FIG. 1) or other known means. Such entities may have appointment scheduling modules or similar known scheduling software that communicates with the scheduling management algorithm 832 and provide feedback to the scheduling management algorithm 832 so that the scheduling management algorithm 832 may confirm that a scheduled appointment is made. Once the patient, the physician, the caregiver, the hospital, as well as the pharmacy provide feedback regarding a certain appointment date and time, the scheduling algorithm then adds this information to the database 120.

The criteria for an appropriate scheduled appointment are determined by the physician requirements, where the physician or possibly the caregiver establishes the scheduling requirements. For example, day of week, time of day, preferred clinic/hospital, as well as which attending medical personnel would be needed.

FIG. 11 is a flow chart illustrating the procedure for setting up an appointment. As discussed above, the appointment scheduling module 830 utilizes a scheduling management algorithm 832 to determine whether or not the pump needs to be refilled or serviced. At step 905, the scheduling management algorithm 832 determines whether an appointment is necessary in accordance with the procedures discussed above. The appointment scheduling module 830 may make this determination either continuously, periodically (e.g., on a daily basis), or even manually (e.g., manually interrogating the implantable device 105 for it's drug status and drug infusion rate conditions).

If an appointment is not required, a record that this determination was made is stored in the database 120 (at step 920). On the other hand, if the scheduling management algorithm 832 determines that an appointment is required, at step 910, the appointment scheduling module 830 schedules an appointment as discussed above and notifies the appropriate parties. In achieving this function, the scheduling management algorithm 832 of the appointment scheduling module 830 may communicate with any number of parties including, but not limited to, a treating physician 155 to alert him/her of the need for a refill, a pharmacy 140 to deliver the necessary drug to the treating physician 155, the caregiver 150 to inform him/her that the patient needs to be taken to the treating physician 155 for a refill, the patient 10, the venue (i.e., hospital or clinic 145) to provide necessary facilities for the treating physician 155 to refill the pump, the manufacturer 130 for the implantable device 105, and the insurance provider 125. Notification of the pharmacy 140 may include, for example, information regarding the type, concentration and quantity of drug to be supplied, the date needed, as well as where the drug should be delivered. At step 915, the treating physician 155 refills or services the pump 105. At step 920, event data (such as the pump refilling and the scheduling parameters) are stored in the database 120. Finally, at step 925, whether or not the pump is refilled or serviced, the scheduling management algorithm 825 requests payment for the scheduling service.

It will be appreciated that the present invention may be implemented using other embodiments. Those skilled in the art recognize that the preferred embodiments may be altered and modified without departing from the true spirit and scope of the invention as defined in the appended claims.

I claim:

1. A computing device in communication with an implantable device for delivering therapy to a patient, the implantable device having a monitor module configured to monitor at least one parameter of the implantable device, the computing device comprising in combination:
(a) a drug scheduling module for determining whether an appointment is necessary to service the implantable device based upon the parameter from the monitor module;
(b) a memory for storing therein the scheduling module and at least one scheduling preference; and
(c) a telemetry module providing bi-directional communication between the computing device and the implantable device.

2. The computing device of claim 1, wherein the drug scheduling module determines whether an appointment is necessary to perform the service selected from the group consisting of a software update, a software modification, a pump refill, and a power supply recharge.

3. The computing device of claim 1, wherein the parameter is a drug usage parameter selected from the group consisting of a quantity drug consumed, a rate in which drug is being consumed, and an estimated date that drug will be exhausted.

4. The computing device of claim 1, wherein the scheduling module receives data from the implantable device, wherein the data is selected from the group consisting of drug usage information, drug management instructions, pump manufacturer requirements, and drug management data.

5. The computing device of claim 4, wherein the drug management instructions is selected from the group consisting of order a drug delivery device refill kit, notify primary care physician of drug order, notify specialty care physician of drug order, notify drug pharmacy to order drug, notify patient's employer of drug order, deliver drug to a specified location, and bill drug to a payer.

6. The computing device of claim 1, wherein the scheduling module receives drug management data selected from the group consisting of name of drug manufacturer, date drug was manufactured, and name of pharmacy carrying the drug.

7. The computing device of claim 1, wherein the scheduling module includes a drug management algorithm to forecast when a next refill of pump reservoir is required.

8. The computing device of claim 1, further comprising an appointment scheduling module for arranging an appointment to service the implantable device.

9. The computing device of claim 8, wherein the appointment scheduling module is capable of contacting at least one entity for the appointment, wherein the entity is selected from the group consisting of a pharmacy, a caregiver, a physician, a hospital, and the patient.

10. The computing device of claim 9, wherein the computing device is operatively coupled to the entity via a computing network.

11. The computing device of claim 10, wherein the computing network is an Internet.

12. An implantable drug delivery device for delivering at least one drug to a patient comprising in combination:
(a) at least one reservoir each containing at least one drug;
(b) a drug scheduling module for determining whether the drug should be replenished;
(c) an appointment scheduling module for scheduling an appointment to replenish the drug in the device: and
(d) a telemetry module providing bi-directional communications with an external device for allowing the scheduling module to schedule the appointment.

13. The implantable drug delivery device of claim 12, wherein the appointment scheduling module contacts via the external device at least one entity for the appointment, wherein the entity is selected from the group consisting of a pharmacy, a caregiver, a physician, a hospital, and the patient.

14. The implantable drug delivery device of claim 12, the drug scheduling module receives data about the implantable drug delivery device, wherein the data is selected from the group consisting of drug usage information, drug management instructions, pump manufacturer requirements, and drug management data.

15. The implantable drug delivery device of claim 14, wherein the drug management instructions is selected from the group consisting of order a drug delivery device refill kit, notify primary care physician of drug order, notify specialty care physician of drug order, notify drug pharmacy to order drug, notify patient's employer of drug order, deliver drug to a specified location, and bill drug to a payer.

16. The implantable drug delivery device of claim 12, wherein the drug scheduling module receives drug management data selected from the group consisting of name of drug manufacturer, date drug was manufactured, and name of pharmacy carryng the drug.

17. The implantable drug delivery device of claim 12, wherein the drug scheduling module includes a drug management algorithm to forecast when a next refill of pump reservoir is required.

18. The implantable drug delivery device of claim 12, wherein the appointment scheduling module is capable of contacting at least one entity for the appointment, wherein the entity is selected from the group consisting of a pharmacy, a caregiver, a physician, a hospital, and the patient.

19. The implantable drug delivery device of claim 18, wherein the computing device is operatively coupled to the entity via a computing network.

20. The implantable drug delivery device of claim 19, wherein the computing network is an Internet.

21. An implantable drug delivery device having a patient scheduling feature, comprising:
(a) a housing;
(b) a drug reservoir carried in the housing configured to contain a therapeutic substance;
(c) a flow control coupled to the drug reservoir for controlling the flow ofthe therapeutic substance from the drug reservoir through an infusion port;
(d) electronics coupled to the flow control and a power source;
(e) a telemetry module coupled to the electronics;
(f) memory coupled to the electronics, the memory containing pump scheduling criteria and other scheduling criteria;
(g) a monitoring module coupled to the memory and the electronics that monitors at least one pump operation variable; and,
(h) a scheduling module coupled to the memory and the electronic, the scheduling module configured to calculate at least one relationship among the pump scheduling criteria, other scheduling criteria, and monitored pump variables, the scheduling module configured to decide whether a pump scheduling activity should be reported, and the scheduling module configured to activate the telemetry module to report a scheduling activity.

22. The implantable drug delivery device of claim 21, wherein the scheduling module determines whether an appointment is necessary to perform a service selected from the group consisting of a software update, a software modification, a pump refill, and a power supply recharge.

23. The implantable drug delivery device of claim 21, wherein the scheduling module communicates via the telemetry module with an external device.

24. The implantable drug delivery device of claim 21, wherein the scheduling module is capable of contacting at least one entity for an appointment, wherein the entity is selected from the group consisting of a pharmacy, a caregiver, a physician, a hospital, and the patient.

25. The implantable drug delivery device of claim 23, wherein the computing device is operatively coupled to the entity via a computing network.

26. The implantable drug delivery device of claim 25, wherein the computing network is an Internet.

27. A method for scheduling activities to support an implantable drug delivery device, comprising:
(a) establishing scheduling criteria;
(b) monitoring pump variables;
(c) calculating at least one relationship among pump scheduling criteria, other scheduling criteria and monitored pump variables;
(d) deciding whether a pump scheduling activity should be reported;
(e) reporting the pump scheduling activity from the implantable drug pump into a communications medium; and,
(f) scheduling the pump scheduling activity with a party.

28. The method of claim 27, wherein the step of deciding includes the step of determining whether an appointment is necessary to perform a service selected from the group consisting of a software update, a software modification, a pump refill, and a power supply recharge.

29. Computer executable instructions for performing the steps recited in claim 27.

30. The method of claim 27, wherein the step of monitoring pump variables includes the step of monitoring at least one drug usage parameter selected from the group consisting of a quantity drug consumed, a rate in which drug is being consumed, and an estimated date that drug will be exhausted.

31. The method of claim 27, wherein the step of establishing scheduling criteria includes the step of obtaining scheduling data selected from the group consisting of drug usage information, drug management instructions, pump manufacturer requirements, and drug management data.

32. The method of claim 31, wherein the drug management instructions is selected from the group consisting of order a drug delivery device refill kit, notify primary care physician of drug order, notify specialty care physician of drug order, notify drug pharmacy to order drug, notify patient's employer of drug order, deliver drug to a specified location, and bill drug to a payer.

33. The method of claim 27, wherein the step of establishing scheduling criteria includes the step of obtaining drug management data selected from the group consisting of name of drug manufacturer, date drug was manufactured, and name of pharmacy carrying the drug.

34. The method of claim 27, wherein the step of deciding whether a pump scheduling activity should be reported is performed by a drug management algorithm.

35. The method of claim 27, wherein the step of scheduling the pump scheduling activity with a party is performed by an appointment scheduling module.

36. The method of claim 27, wherein the step of scheduling the pump scheduling activity includes the step of contacting the party selected from the group consisting of a pharmacy, a caregiver, a physician, a hospital, and the patient.

37. The method of claim 36, wherein the step of contacting is performed via a computing network.

38. The method of claim 36, wherein the step of contacting is performed via an Internet.
